(19) United States
(12) Patent Application Publication
(10) Pub. No.: US 2009/0149782 A1
(43) Pub. Date: Jun. 11, 2009

(54) Non-Invasive Neural Stimulation

(76) Inventor: Donald Cohen, Irvine, CA (US)

Correspondence Address: FISH & ASSOCIATES, PC;ROBERT D. FISH, 2603 Main Street, Suite 1000, Irvine, CA, 92614-6232, US

(21) Appl. No.: 12/333,203

(22) Filed: Dec. 11, 2008

Related U.S. Application Data
(60) Provisional application No. 61/012,851, filed on Dec. 11, 2007.

(51) Int. Cl. A61N 7/00; A61N 1/00

(52) U.S. Cl. 601/2; 607/115

(57) ABSTRACT

The present invention is a system for the non-contact stimulation of excitable tissue. A primary purpose is reducing the perception of pain in those people who suffer from persistent pain. Apparatus is described for adjusting the position of the stimulation region.

This application claims the benefit of priority to U.S. Provisional Application having Ser. No. 61/012,851 filed on Dec. 11, 2007

### FIELD OF THE INVENTION

The field of the invention is non-contact stimulation of excitable tissue. Particular applications include neural stimulation for the treatment of pain, migraine, angina symptoms, urinary incontinence, and activation of muscular contraction.

### BACKGROUND

In order for pain to be perceived, a neural signal generally travels along an afferent nerve from the site of the pain, through the spinal cord and up to the brain. If the signal is inhibited, interrupted or “confused” along the way, the pain is relieved to some extent or replaced by paresthesia or tingling.

Neurons are specialized excitable cells that can transmit electrical signals via ionic transport across their membranes. The energy needed to initiate the excitement of the nervous tissue can be any of several different forms. Biologically, this ionic transport (depolarization) is generally initiated by chemical energy. This initiates depolarization at a synapse between neighboring neurons. Neurotransmitters released by one neuron can initiate the depolarization in the next neuron. In sensory afferent neurons the initiation energy can be one of many different forms of energy. It can be photons (as in the retina of the eye), mechanical air pressure (as in the ear), mechanical force (as in the skin), etc.

Within the first couple of mm of skin, there are several types of neurons specialized to be particularly susceptible to excitation by a particular mechanical stimulus. Below are some examples of the neurons, their typical skin depths and the particular sensitivity.

TABLE 1

| | | |
|---|---|---|
| Meissner corpuscle | 0.7 mm | Low frequency vibration |
| Merkel cell | 0.9 mm | Pressure |
| Ruffini ending | 1.5 mm | Lateral extension |
| Pacinian corpuscle | 2.0 mm | High frequency vibration |

The threshold energy needed to initiate depolarization is most efficient for the particular form associated in the preceding list. Each neuron can be stimulated by different energy expressions, but is most suited to a particular one.

In the case of neural stimulator devices the initiating energy is electrical. A voltage is applied between electrodes that are placed in the vicinity of nervous tissue that is intended to be stimulated. Current flows between the electrodes and through the nerve cells. When the electrical energy is above a certain threshold, the nerve cells in that region will be depolarized.

Depolarization is also achievable using ultrasound energy even if the threshold energy level for ultrasound is higher than for electrical stimulation. Similarly, axonal stimulation (along the length of the neuron rather than at the end suited for stimulation) is possible though not as efficient. In other words, a greater intensity will be needed to stimulate neurons in the middle.

### Invasive Spinal Cord Stimulation

Many people who suffer from intractable chronic pain achieve a measure of relief from the use of implantable spinal cord stimulation systems. Generally, leads containing multiple electrodes are implanted on top of the dorsal surface of the spinal cord and connected to a pulse generator/processor located several inches away. Electrode stimulation configuration is chosen to optimize the effectiveness of the stimulating pulsations. More information about neuron-stimulation equipment and how it works can be found at the following:
http://www.medscape.com/viewarticle/554863
http://www.webmd.com/back-pain/guide/spinal-cord-stimulation
http://www.medtronic.com/servlet/ContentServer?pagename=Medtronic/Website/Condition Article&ConditionName=Chronic+Back+and%2For+Leg+Pain&Article=bpain_art_nsproducts
http://www.controlyourpain.com/index.cfm?langid=1
http://www.ans-medical.com/

### Ultrasonic Stimulation of Muscle

For relief of muscle pain, unfocused ultrasonic therapy has been used to promote healing and relief of pain.

### Ultrasonic Stimulation to Promote Bone Healing

Ultrasound has been used to promote bone healing. More information may be found at http://ortho.smith-nephew.com/us/node.asp?NodeId=2865. Excerpted:

“Treatment of fractures with the EXOGEN™ 4000+* Bone Healing System (low-intensity pulsed ultrasound) may speed healing, lower the need for further surgery, and get patients back to their normal activities faster . . . . The EXOGEN™ Bone Healing System utilizes low-intensity ultrasound to accelerate the healing of indicated fresh fractures up to 38% faster than normal healing. The Exogen™ Bone Healing System is also highly effective for use on non-healing fractures. The ultrasound device is a portable, lightweight unit that delivers the prescribed treatment in a convenient 20 minute daily regimen . . . treat themselves at home, thus freeing hospital resources from this task. The treatment is safe and has no contra-indications. It has been clinically proven in many thousands of patients worldwide.”

Exogen U.S. Pat. No. 6,432,070 Exogen, Inc. (Piscataway, N.J.), and all other extrinsic materials discussed herein, are incorporated by reference in their entirety. Where a definition or use of a term in an incorporated reference is inconsistent or contrary to the definition of that term provided herein, the definition of that term provided herein applies and the definition of that term in the reference does not apply. Also, unless a contrary intent is apparent from the context, all ranges recited herein are inclusive of their endpoints, and open-ended ranges should be interpreted to include only commercially practical values.

### Ultrasonic Neurolysis for Pain

Another way to address unrelenting, unmanageable pain (as in cancer patients) is to irreversibly destroy nerves associated with the pain. High Intensity Focused Ultrasound has been used to destroy nerve cells in the interest of relieving pain. See for example
http://depts.washington.edu/bioe/people/core/vaezy/vaezy.html or U.S. Pat. No. 7,305,264 or application 20060184069.

Other techniques have also been used to destroy nerve cells for this purpose. See for example Critical Evaluation of Chemical Neurolysis from Cancer Control: Journal of the Moffitt Cancer Center http://www.medscape.com/viewarticle/408975_{—}3.

### Ultrasonic Stimulation in Tactility Research

It has been shown that ultrasonic energy can stimulate nerves and cause tactile sensations. See for example http://www.biomedical-engineering-online.com/content/2/1/6 Can ultrasound be used to stimulate nerve tissue? Stephen J Norton, BioMedical Engineering OnLine 2003, 2:6doi:10.1186/1475-925X-2-6 and http://www.alab.t.u-tokyo.acjp/˜shino/research/pdf/icat01.pdf ICAT Dec. 5-7, 2001 Tokyo, JAPAN Focused Ultrasound for Tactile Feeling Display, Takayuki IWAMOTO, Taro MAEDA, and Hiroyuki SHINODA, The University of Tokyo.

Furthermore, it is well known that ultrasound can be transmitted into the human body, as illustrated for example in figures at the website http://www.sprawls.org/ppmi2/USPRO/.

Accordingly, ultrasound can be transmitted to non-superficial tissues to stimulate nerves.

### TENS

Transcutaneous Electrical Nerve Stimulations (TENS) is a safe non-invasive drug-free method of pain management. Such units relieve pain by sending small electrical impulses through electrode pads placed on the skin to underlying nerve fibers http://www.vitalityweb.com/backstore/tenswork.htm

There are several proposed explanations for how TENS may work:
electrical stimulation of the nerve fibers block a pain signal from being carried to the brain
activation of the release of natural chemicals called endorphins in the brain which act as analgesics
stimulation of the nerves that perceive pain or light touch
interference with nerve pathways
effects on flow of vital energy (used to explain acupuncture) have also been offered to explain TENS
TENS may affect the cardiovascular system, increasing heart rate and reducing blood pressure
http://www.intelihealth.com/IH/ihtIH/WSIHW000/8513/34968/363973.html?d=dmtContent#background.

### Non-Invasive Magnetic Stimulation

Transcranial magnetic stimulation (TMS) has been used to stimulate neurons, particularly in the brain. It uses very strong magnetic fields to create electrical fields that can stimulate neurons. The field strength decays rapidly with distance, so TMS is only used to create cortical stimulation—not deep brain stimulation. http://www.neuronetics.com/default.asp

### Limitations of Present Technologies

When using electrodes to deliver stimuli to excitable tissue, electrical voltage gradient generally diminishes with distance from electrodes. It is very difficult to stimulate a target nerve without also stimulating many unintended nerves and muscles too. The nerves that are closest to the electrodes generally experience the highest voltage gradient, highest current flow, and are thus the most likely to be stimulated by the electrical pulse. The location of supra-threshold pulses can be adjusted by varying the location, quantity of and voltage of or current from the electrodes—but the supra-threshold region is always located between or very close to the electrodes.

Non-invasive electrical and unfocused ultrasonic neural stimulators do not do a good job of stimulating only the target nerves. The selection of nerves stimulated by the implantable system can be influenced by the placement of the electrodes and by selection of which electrode combination is actually utilized. Though this does a much better job of limiting the stimulating to the desired tissue, it has the considerable drawback of requiring invasive surgery.

Another drawback of implantable electrodes and leads is the eventual loss of effectiveness. This is often attributable to migration of electrodes with respect to the target nervous tissue. It is sometimes attributable to the necrosis of nervous tissue due to prolonged mechanical stress imposed by the electrodes and/or leads. Effectiveness can sometimes be re-established by changing the characteristics of the electrical pulses or by using different electrode combinations. In the extreme, the electrodes can be invasively re-positioned.

Loss of effectiveness can also be caused by an increase of excitability threshold of the target tissue. This degradation of tissue can be a response to the physical presence of the electrodes and leads and to the associated stresses.

Focused ultrasound has been used to destroy tissue and thus block pain. This has the drawback of not being reversible. Neural stimulation can be reversed, but neurolysis is essentially permanent. Targeting mistakes in are thus much more severe in focused ultrasound neurolysis than in neuro stimulation.

Thus, there is still a need for methods and devices that stimulate target nervous tissue without simultaneously inadvertently stimulating appreciable quantities of intervening and neighboring nerve and muscle tissue; and to do so without the need for invasive surgery. There is also a need to manipulate the position of the subcutaneous supra-threshold stimulus non-invasively, and to avoid degradation, necrosis or lysis of excitable target tissue.

It is an objective of various embodiments the current invention to deliver stimuli to target excitable tissue without direct contact, and without undue stimulation of other intervening or neighboring excitable tissue. It is a further objective of such embodiments to have the capability to adjust the location and timing and magnitude of the stimuli. It is a still further objective of such embodiments to have the capability of automatically adjusting the characteristics of the stimuli, including pulse strength, timing and location.

### Additional Prior Art Information

Weblinks
http://www.medscape.com/viewarticle/554863
http://www.webmd.com/back-pain/guide/spinal-cord-stimulation
http://www.medtronic.com/servlet/ContentServer?pagename=Medtronic/Website/ConditionArticle&ConditionName=Chronic+Back+and%2For+Leg+Pain&Article=bpain_art_nsproducts_m
http://www.controlyourpain.com/index.cfm?langid=1
http://www.ans-medical.com/
http://ortho.smith-nephew.com/us/node.asp?NodeId=2865
http://depts.washington.edu/bioe/people/core/vaezy/vaezy.htmlCancer Control: Journal of the Moffitt Cancer Center
http://www.vitalityweb.com/backstore/tenswork.htm
http://www.intelihealth.com/IH/ihtIH/WSIHW000/8513/34968/363973.html?d=dmtContent#background
http://www.harfangmicro.com/FAQ.html
http://www.bioe.psu.edu/ultrasound/research/Saleh%20Smith%20IJH04.pdf
http://www.olympusndt.com/data/File/intro_pa/Intro_PA_Chap1.en.pdf
http://www.radiologyresearch.org/SPI01-MI4325-51.pdf
http://www.victhom.com/en/realization-neurostep-8.htm
http://www.neuronetics.com/

Journal Articles
http://www.medscape.com/viewarticle/408975_{—}3
http://www.biomedical-engineering-online.com/content/2/1/6 Can ultrasound be used to stimulate nerve tissue? Stephen J Norton, BioMedical Engineering OnLine 2003, 2:6doi:10.1186/1475-925X-2-6
http://72.14.253.104/search?q=cache:-4JFY1BIEwoJ:www.alab.t.u-tokyo.ac.jp/˜shino/research/pdf/icat01.pdf+focused+ultrasound+for+tactile+feeling+display&hl=en&ct=clnk&cd=l&gl=us ICAT Dec. 5-7, 2001 Tokyo, JAPAN Focused Ultrasound for Tactile Feeling Display, Takayuki IWAMOTO, Taro MAEDA, and Hiroyuki SHINODA, The University of Tokyo
A. B. Valbo: Properties of cutaneous mechano receptors in the human hand related to touch sensation, Human Neuro Biology, 3, pp. 3-14 Springer-Verlag, 1973

US Patents

| | | | |
|---|---|---|---|
| 6,432,070 | 5,762,616 | 4,889,526 | 4,800,898 |
| 7,011,638 | 4,530,360 | 4,541,432 | 4,535,777 |
| 6,206,843 | 6,652,443 | 4,510,936 | 4,343,301 |
| 5,413,550 | 3,828,769 | 7,081,128 | 7,367,956 |
| 6,748,275 | 6,091,994 | 7,033,312 | 7,305,264 |
| 6,721,603 | 6,066,084 | 5,983,141 | 7,369,894 |
| 5,460,595 | 5,807,285 | 5,766,124 | |
| 5,556,372 | 5,476,438 | 4,940,453 | |

US Patent Applications 20060184069

### SUMMARY OF THE INVENTION

The inventive subject matter provides apparatus, systems and methods in which a non-contact device stimulates excitable tissue beneath the skin without causing unduly uncomfortable stimulation of intervening tissue. The purpose of the stimulation can be for the relief of the perception of pain, for stimulating tissue to contribute to urinary continence, for behavior modification, or for stimulating other tissue.

### BRIEF DESCRIPTION OF DRAWINGS

FIG. 1a shows a representation of a front view of transducer head containing a number of individual transducers, each transducer depicted with a beam (depicted as translucent) emanating from it. The transducers are mounted along two crossing different radius curves. The beams emanating from the transducers parallel to the front plane can be seen to cross at a focal point beneath the transducer head.

FIG. 1b shows a representation of a side view of the transducer head of FIG. 1a containing a number of individual transducers, each transducer depicted with a translucent beam emanating from it. The beams emanating from the transducers parallel to the side plane can be seen to cross at a focal point beneath the transducer head; this point being deeper than the focal point evident in FIG. 1a.

FIG. 1c shows a representation of an isometric view of the transducer head of FIGS. 1a and b containing a number of individual transducers, each transducer depicted with a translucent beam emanating from it. It can be seen that the half of the beams cross at a shallow focus; the other half intersect at a deeper focus.

FIG. 2a shows a representation of a front view of transducer head containing nine individual transducers. The transducers are mounted so that the centerline of each one crosses directly through target focal points represented as small spheres. In the configuration depicted in this figure, three of the nine each transducer are depicted with a translucent beam emanating—each beam representing ultrasonic energy emanating from each transducer. These three beams can be seen to cross at the bottom target focal point.

FIG. 2b shows a representation of the bottom view of the transducer head depicted in FIG. 2a containing nine individual transducers.

FIG. 2c shows a representation of an isometric view of the transducer head depicted in FIGS. 2a and b.

FIG. 3 shows a representation of an isometric view of the transducer head depicted in FIG. 2, in which a translucent beam is depicted as emanating from each. At each of the 5 spheres, three of the beams intersect.

FIG. 4a shows a representation of the front view of the transducer head of FIGS. 2 and 3 with the housing removed to allow a more direct view of the transducers and the three beams that intersect at the top point.

FIG. 4b shows a representation of the front view of the transducer head of FIG. 4a with the three beams that intersect at one of the intermediate points depicted as translucent.

FIG. 4c shows a representation of the front view of the transducer head of FIGS. 4a and b with the three beams that intersect at the bottom point depicted as translucent.

FIG. 4d shows a representation of the front view of the transducer head of FIGS. 4a, b and c with translucent beams depicted as emanating from each of the nine transducers. Three beams intersect at each of the five points.

FIG. 5a shows a representation of an isometric view of the transducer head of FIG. 4a with the housing removed to allow a more direct view of the transducers and the three beams that intersect at the top point.

FIG. 5b shows a representation of an isometric view of the transducer head of FIG. 4b with the three beams that intersect at one of the intermediate points depicted as translucent.

FIG. 5c shows a representation of an isometric view of the transducer head of FIG. 4c with the three beams that intersect at the bottom point depicted as translucent.

FIG. 6a shows an isometric view of a gimbal mounted focused transducer; one lever that controls rotations about an axis, a second lever that controls rotation about a second perpendicular axis; and a third that allows for helical rotation to adjust travel along the third axis that is orthogonal to that established by the plan of the prior two axes.

FIG. 6b is an isometric view of the gimbal mounted focused transducer of FIG. 6a, shown in partial section—affording a better view of a curved focused transducer within.

FIG. 7 shows a chart representing stimulation pulse train envelopes transmitted to the excitable tissue.

FIG. 8 is a block diagram of a focused ultrasonic neural stimulator system.

FIG. 9a is a representation of a prior art technology, ultrasound phased array beam intensity.

FIG. 9b is a representation of a prior art technology from Olympus NDT, ultrasound phased array, used to form a focal point of ultrasound energy; depicted showing the wavefronts at four times after the wavefronts have left the transducers; the bottom ones showing how they meet at a central focal point.

FIG. 9c is a representation of a prior art technology from Olympus NDT, ultrasound phased array, used to form a focal point of ultrasound energy; depicted showing the wavefronts at four times after the wavefronts have left the transducers; the bottom ones showing how they meet at an eccentric focal point.

FIG. 9d graphically shows the delay values used in a 32 element ultrasound phased array that are used to create different focal depths.

FIG. 9e illustrates different focal depths that are created below a 32 element ultrasound phased array by using the delay values of FIG. 9d.

FIG. 10a illustrates the intensity (as determined by Shafiri et. al. of the University of Tehran) that can be achieved at a single focus using a planar array of ultrasound elements.

FIG. 10b illustrates on possible orientation of ultrasonic transducers in a planar phased array.

### DETAILED DESCRIPTION OF THE INVENTION

Patent Application 61/012,851 is included by reference.

### Supra-Threshold Spot Adjustment

Without desiring to be held to any particular theory or mechanism of action, it is currently contemplated that stimulation (or capture) of the correct tissue is very important to eliciting the desired response. It is also contemplated that another important factor is doing so without stimulating too much neighboring or intervening tissue. Preferred embodiments of the present invention achieve this by creating a supra-threshold high intensity region remote from the energy emitter, and the energy intensity in the intervening region is sub-threshold. The supra-threshold region is located several millimeters away from the energy emitter interface with the body. In especially preferred embodiments this is accomplished with focused ultrasound energy or with overlapping or interfering ultrasound beams. The location of this supra-threshold spot must be adjustable—upon initial placement and on occasion when there is need to reposition due to loss of capture, or when there is need for optimization or when there is a desire to stimulate other tissue. This adjustment can be done electrically, mechanically, electromechanically or equivalent.

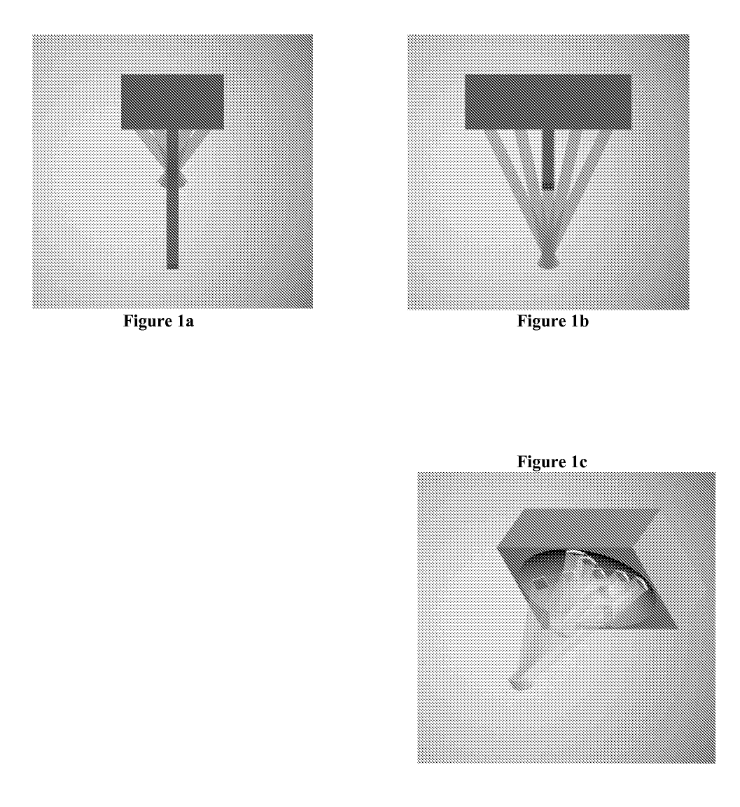

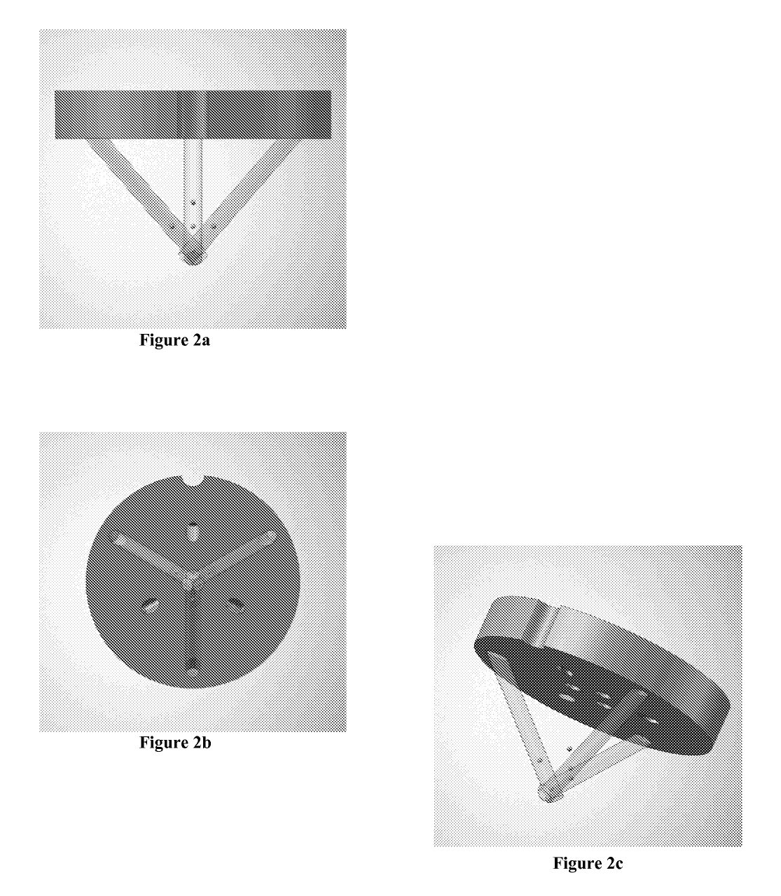

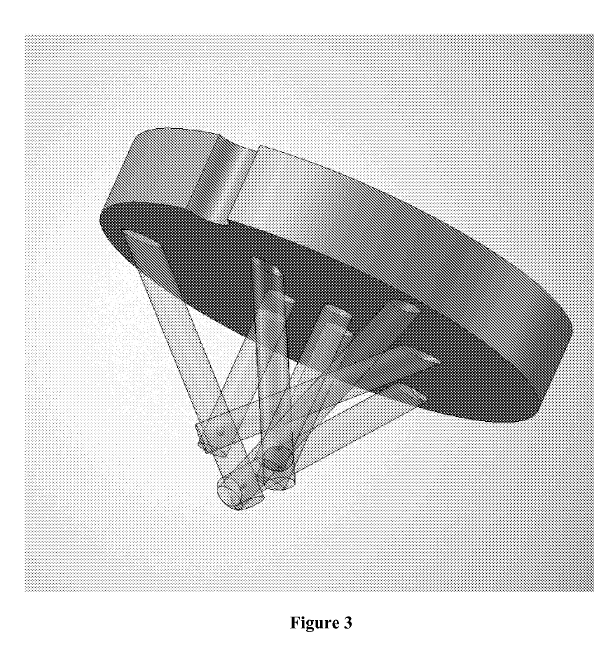

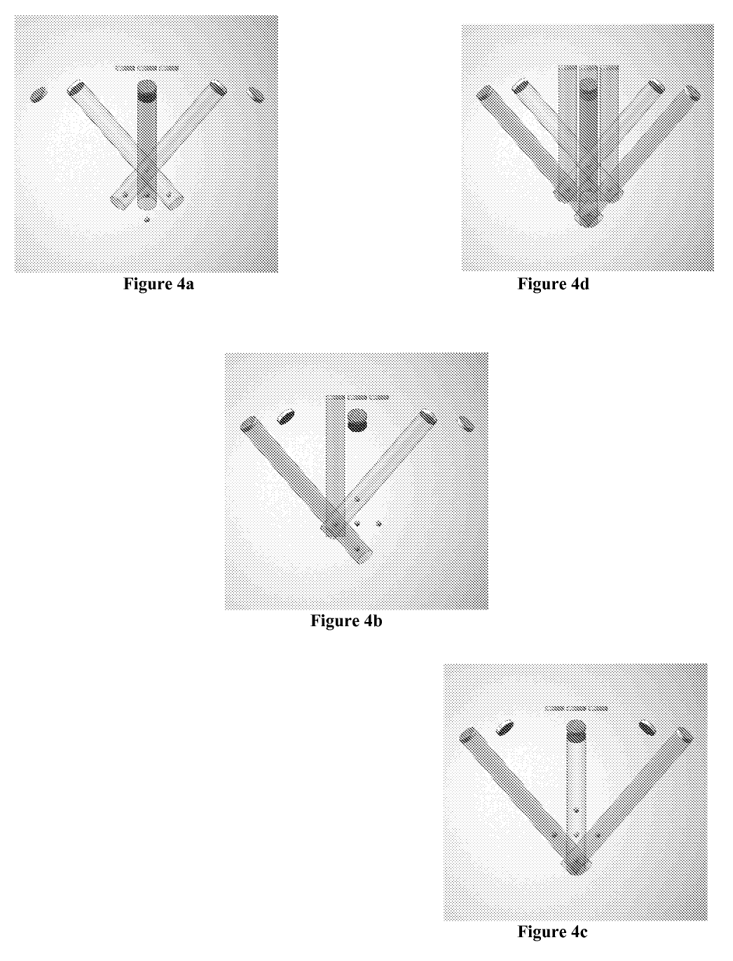

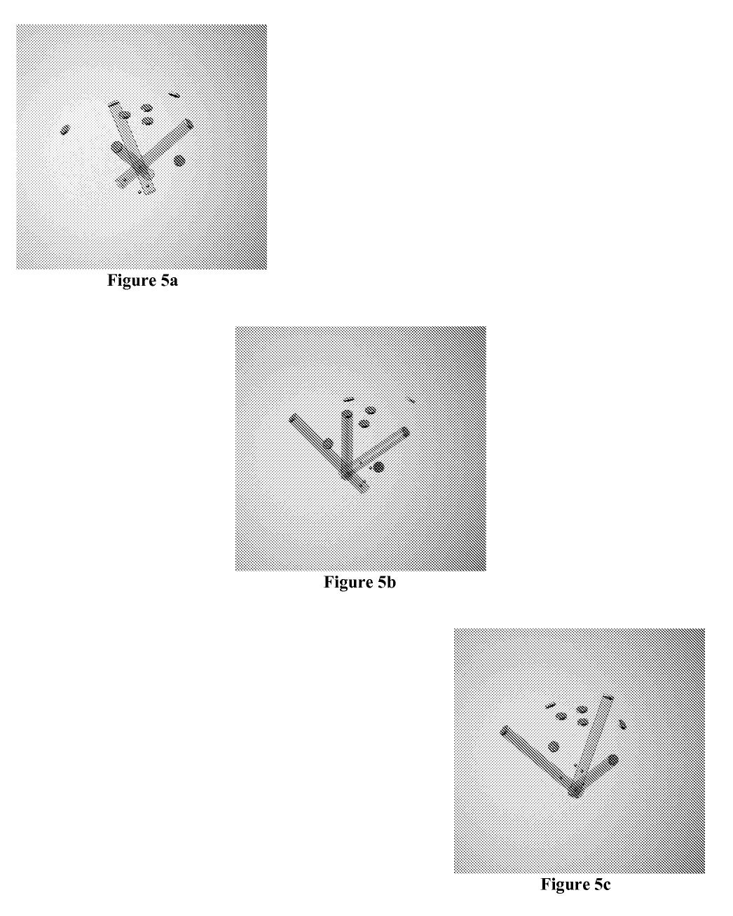

Electrical adjustment of the high intensity spot position can be done by choosing which group of ultrasound crystals to energize from among several to create a high intensity region achieved by overlapping beams as illustrated in FIGS. 1 through 5. These figures illustrate a transducer head which can be placed on the skin close to directly over the target tissue. The transducer housing is placed as accurately as possible so that the high intensity spot is close to right over the target tissue—likely to be 10 to 20 mm beneath the skin surface.

In these figures, nine discrete crystals are used—selecting the proper combination of three of these allows the user to select from among five points that surround a central location. These neighboring regions can be overlapping. One point is directly above, and one point is directly below the central target. The other 3 points are in a plane between the upper and lower point, and they surround the center. Since the five points are neighboring, perhaps even overlapping, the high intensity region can be moved up or down, side to side or forward or back without moving the transducer head mechanically at all.

Alternatively, the electrical spot location adjustment could use a different number of overlapping beams. Using just two beams makes it practically simpler to define many more high intensity spots—though there would not be as distinct and abrupt intensity differentiation from surrounding tissue. Using more overlapping beams improves the resolution of the high intensity region—i.e. the overlapping region has a much higher intensity than the environs and therefore there is even less likely to be inadvertent ancillary stimulation.

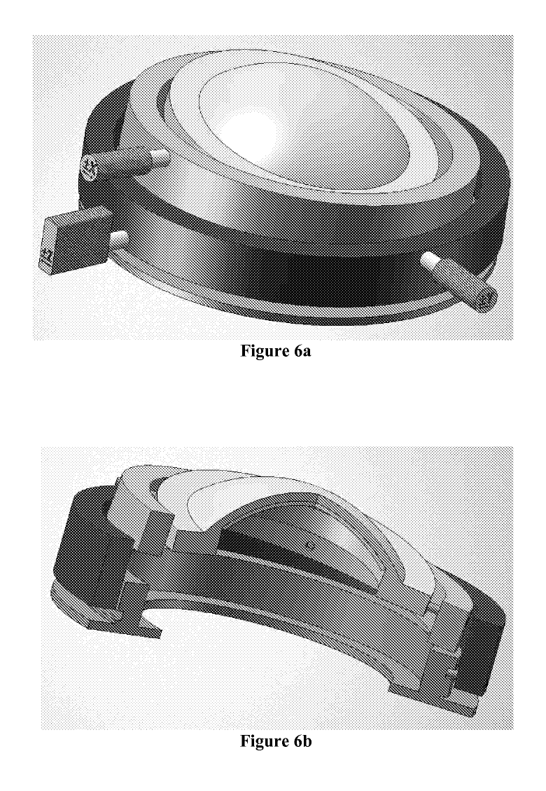

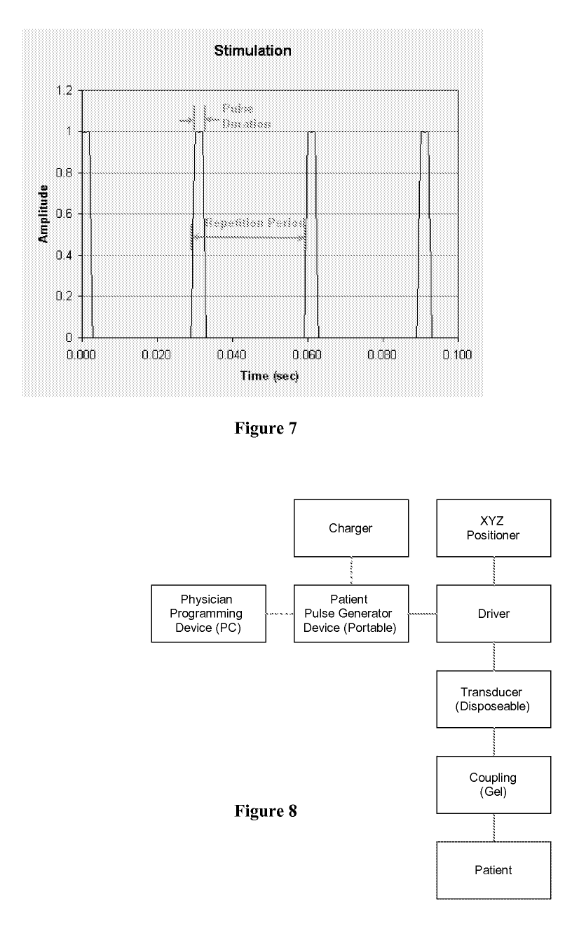

Other embodiments for creating an adjustable high intensity spot mechanically move an ultrasonic focal spot. An example of this is the gimbal mounted focused ultrasonic crystal depicted in FIG. 6. The high intensity region is at a fixed distance from the crystal. This spot is adjusted by the gimbal mechanism—and can be adjusted by the patient or the practitioner. There are three controls which allow adjustment in each of the three principal directions to achieve nearly infinitely variable location of the focal spot. The gimbal is adjusted by independent rotation of levers that control rotation about two perpendicular axes, and by a third adjustment that uses a helical track to adjust height. Rather than a gimbal, the adjustment can be accomplished by other equivalent apparatus such as x, y, z positioner.

The gimbal can be designed to allow Cartesian (x, y, z), polar (θ, φ, r) or other equivalent positional translation. The adjustment controls can be mounted (i) directly on the gimbal mount, or alternatively (ii) mechanically coupled though located remotely, as a joystick at the end of a cable for easier patient control, or alternatively (iii) located remotely, by radio control of three separately addressable motors, or (iv) an equivalent control system.

Another alternative to allow for adjustment of the high intensity supra-threshold spot is a variable focus mechanism instead of a fixed focus one. Some of the ways to achieve the variable focus are: the spacing of multiple focusing elements can be adjusted, the density of the lens can be adjusted, the shape of the lens can be adjusted, the location of elements of the transducer can be adjusted, or equivalent.

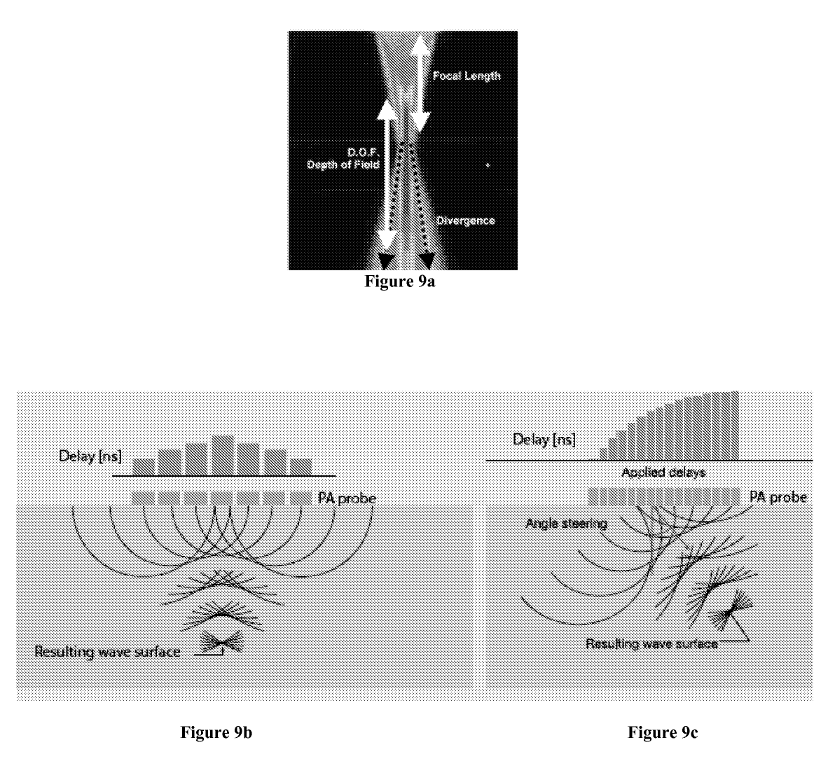

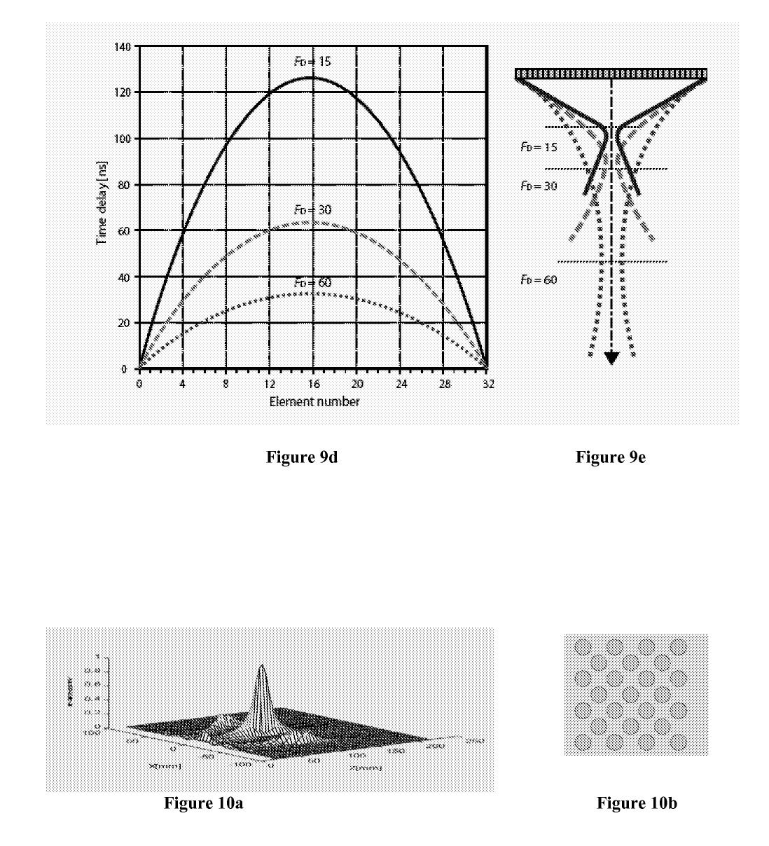

Another alternative to create an ultrasound focus is to use phased array technology as depicted in FIG. 9. Not only can this technology be used to create a focus, but it can also be used to move the focus up or down, forward or back, left or right within a zone beneath the array. FIG. 9b depicts how a phased array can be used to pulse each crystal using variable, but symmetric timing to form a focal point directly beneath the center of the array. FIG. 9b depicts how the crystals of the same array can be pulsed with eccentric delays to form a focal point that is located eccentrically. Using similarly “shaped” delays of different magnitudes, the focus can be shifted variably up or down as well as left or right. FIGS. 9d and e illustrate how different delays can shift the focal depth deeper or shallower.

Using additional array elements positioned in a different plane, e.g. an orthogonal plane, a shaped phase delay can shift the focus in an orthogonal direction. Adjusting the delays to each of the crystals within such an array will allow essentially infinitely variable focal adjustment beneath the array.

Use of a phased array of multiple elements arranged on a surface (rather than just in a line) as in FIG. 10 allows for movement of the focus left or right, up or down and forward or back. Within a volume underneath the surface array, the focus can be adjusted to be virtually anywhere. The array surface may be flat or not.

In yet another alternative embodiment of an array of energy emitters to create a focal region that can be adjusted, the array elements would be electrodes rather than ultrasonic emitters. In a similar manner, the timing of the application of voltage to each electrode would be adjusted to allow for creation of a region of high intensity at a location remote from the electrode array. Ideally, the timing of the application of voltage to each electrode would be adjusted to allow for adjustment of the location of the high intensity, supra-threshold region in space; ±X, ±Y, ±Z.

### Automatic Adjustments

During the course of operation, it is likely that the stimulation of the target spot becomes compromised. The desired tissue stimulation may no longer be achieved because of threshold change or positional change. Either way—the pulsing that was once effective would be effective no longer. Adjustments could be made to spot location or pulse characteristics to recapture the target tissue. The adjustments could be made by the patient or by a practitioner or by the neuro-modulation system. It could be most convenient, prompt and accurate if the adjustments to spot location and pulse characteristics were done automatically by the system—transparent to the patient.

In order to make the adjustment automatically, it is necessary to be able to detect capture of the target tissue—i.e. a sensor that is an indicator of efficacy. The indicator could be an action potential sensor, an EMG or equivalent. One example of such a capture sensor is an electromyogram (EMG). As an example, a patient experiencing pain is likely to feel tense. This tension would often be expressed as contraction of muscles; and this muscle contraction can be detected by EMG, preferably non-invasively. Relief of the pain by successful capture could be expressed as relaxation of the contraction of indicator muscles. The effectiveness of the neuro-stimulation would result in relaxation of the muscles, and this would be reflected in the EMG sensor.

Electrical characteristics of the pulse can be modified while monitoring the indicator EMG. A decline in the indicator EMG frequency is an indication of successful capture.

Similarly, the system can alter the position of the high intensity pulse while monitoring the indicator EMG; decline in EMG frequency indicates successful capture of the right tissue.

The muscle group to serve as source of the EMG indicator could be individualized for each patient. The muscle group could be located near to the location of the perception of the pain source. For example, in a patient that experiences pain radiated in the foot, the EMG electrodes could be place on the foot. Alternatively, a more general selection—such as the trapezius muscle may be a good indicator for most patients. Relaxation of this muscle would be an indication that the perception of pain has subsided—and that the stimulation parameters are adequate.

The feedback could be binary or analog. In other words, in a binary system, feedback could be used to indicate whether the neurostimulation system has achieved capture or has failed to capture. In an analog system, the feedback would be used qualitatively to indicate how effective the stimulation treats the symptoms.

After manual determination of a baseline threshold, location and adequate sensor for feedback, the automatic adjustment process can be initiated. Capture detection can be automatically checked and adjusted periodically—for example once every 5 minutes. To check for capture and for optimization, each of the following parameters could be incremented to check for improvement of degradation of performance as indicated by the sensor response:

Ultrasound Power

Ultrasound Intensity

Ultrasound Frequency

Ultrasound Waveform

Pulse Envelope Shape

Pulse Duration

Pulse Repetition Rate

X position

Y position

Z position

This adjustment of aim and intensity of the stimulating waveform could be performed frequently to allow for refinement in stimulation in response to patient position, activity level, sympathetic tone or acute intensification of perceived pain.

### Settings

The settings of energy, timing and position may be adjusted within a very large range. In a preferred embodiment, the energy source is ultrasonic; the peak power is 10 watts; the power intensity in the high intensity region is 10 the fundamental resonant frequency is 1 MHz; the repetition rate is 50 Hz; the pulse duration is 2 milliseconds; the focal point is 15 mm sub-dermal.

The waveform may be a simple sine wave or a complex waveform. The envelope of the repetition pulsing may be square or more complex. The amplitude could ramp up or down for example during the course of a pulse.

The pulsing frequency is chosen so that there is enough transmission so that there is enough penetration into the flesh to the desired target level. It is also chosen so that enough energy is absorbed so that there is tissue excitation.

The system may be used continuously to stimulate; alternatively, the system is quiescent for periods. The timing of stimulation and quiescence may be programmable. Ideally, the stimulation is maximized during periods when especially needed, for example when trying to get to sleep. It would be minimized when not needed as much, for example when the patient is already asleep. It may be programmed automatically to be quiescent for periods, such as for 40 minutes of each hour.

Though the stimulation has been described as fairly regular, it need not be. The pulse duration may be variable for example. Particular pulse durations and pulse duration intervals may be suited to particular applications.

### Applications

The non-contact neural stimulation device invention can be used for any of several different applications. It can be used in the treatment of pain in a manner similar to spinal cord stimulation (SCS). Just as with SCS, supra-threshold stimuli can be delivered to neural tissue to create a tingling sensation that blocks or inhibits the perception of pain. The current invention has several significant advantages and some limitations with respect to SCS. One major advantage is that it does not mandate invasive surgery. For this application, the ultrasonic transducer would be located on the skin of the back near the spinal cord. The bony structures of the spine partially obscure ultrasonic energy access to the nerves within the cord—but there is still access.

SCS generally stimulates the nerves along the dorsal horn of the spinal cord. Stimulation of deeper nerves is not practical with SCS unless the SCS lead and electrodes are placed within the cord itself (entailing extra risks and complications). An important advantage of the present invention is that it is not limited to stimulating only the most dorsal surface of the spine. The present invention may be used to stimulate within the spinal cord without stimulating the dorsal surface. This makes possible many other neural stimulation targets that are not typically accessible by SCS.

Targeting neural stimulation targets within the spinal cord can be even more precisely achieved with the ultrasonic transducer placed even closer to the target nerves. In one embodiment, the ultrasonic transducer is implanted within the body close to the spinal cord. A high intensity supra-threshold region is created in front of the transducer spaced apart from it. This invasive application of the present invention allows for more precise targeting of excitable tissue than SCS does. A focused ultrasonic transducer of the present invention can create a supra-threshold region more precisely than the SCS electrical stimulation. The supra-threshold spot can be smaller and the intensity relative to the surrounding tissue can be more dramatic compared to SCS.

The present invention may also be used to target excitable tissues in other areas for other applications. It many be used to excite nerve roots near to where they exit the spinal cord. It may be used to excite peripheral nerves for applications analogous to peripheral nerve stimulation. These include treatment of craniofacial neuropathic pain or restoration of motor functions in patients who have experienced stroke or spinal cord injury. Stimulation of the occipital nerve for example is a way to treat migraine headaches. Other applications include treatment of angina symptoms, urinary incontinence, etc.

Because the present invention can stimulate excitable tissue remotely, it may be used to stimulate cardiac tissue. It can be used to pace the sinoatrial node, the atrioventricular node, myocardial tissue or equivalent. It could be useful as a way to quickly, easily and non-invasively provide emergency cardiac pacing.

Other applications include stimulation of other anatomical structures. An example is stimulation of excitable structures associated with the stomach and other organs of digestion to elicit a sensation of satiety for the purpose of bariatric treatment.

Other applications include stimulation of other nerves for systemic influence. Cardiac, Vagus or other nerves can be stimulated. Applications could be treatment of anxiety, depression, hypertension, etc.

Thus, specific embodiments and applications of non-invasive neural stimulation have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. An apparatus for stimulation of tissue within the body of a mammal to a threshold of excitement, said apparatus comprising a plurality of energy emitters that cooperate to create a supra-threshold region of high energy intensity within the body remote from the emitter, and an intervening sub-threshold region of at least 2 mm in depth.

2. The apparatus of claim 1, wherein at least one of the energy emitters emits ultrasonic energy.

3. The apparatus of claim 2, wherein the energy emitters compose an array selected from the list consisting of:
a linear phased array energy emitter;
a planar phased array energy emitter;
a non-planar surface of phased array energy emitters;
a single element lens focused ultrasound energy emitter;
a multi-element lens focused ultrasound energy emitter;
at least two superimposed ultrasound emitters; and
at least two non-planar ultrasound energy emitters directed at the same region.

4. The apparatus of claim 1, further comprising an adjustment control that operates to position the remote high energy intensity region.

5. The apparatus of claim 4, wherein the adjustment control is a mechanism chosen from the list consisting of:
a mechanical gimbal apparatus;
a mechanical gimbal apparatus connected to a mechanism that allows the gimbal to be adjusted from a location remote from the gimbal;
an electromechanical apparatus;
an electromechanical apparatus with a mechanism that allows position adjustment from a remote location;
a lens position adjustment mechanism;
a lens shape adjustment mechanism;
a phased array adjustment; and
at least two groups of energy emitters, the energy of each group directed to converge at a distinct region, and adjustment of position accomplished by selection of one of the groups.

6. The apparatus of claim 4, further comprising an array of electrodes, and an electrical energy source that supplies voltage to each electrode.

7. The apparatus of claim 2, wherein at least half of the energy emission of the ultrasonic energy emitter is between 20 kHz and 5 MHz.

8. The apparatus of claim 2, wherein the ultrasonic energy emitter is pulsed at a repetition rate between 1 and 1000 Hz, for a duration between 0.01 and 10 milliseconds.

9. The apparatus of claim 1, wherein the region of high energy intensity is between 1 and 100 mm from the at least one energy emitter.

10. The apparatus of claim 1, wherein the high intensity region is at a power intensity between 0.1 and 50 watts/cm².

11. The apparatus of claim 2, wherein the ultrasonic energy emitter is operated with a waveform so that at least 20% of energy emitted is transmitted 20 mm into the body.

12. The apparatus of claim 2, wherein the ultrasonic energy emitter is situated outside the body.

13. The apparatus of claim 2, wherein the ultrasonic energy emitter is implanted within the body.

14. The apparatus of claim 1, further comprising a sensor disposed to detect capture.

15. The apparatus of claim 14, further comprising a mechanism that adjusts a setting of the apparatus in response to a value of the sensor.

16. The apparatus of claim 15, wherein the setting is selected from the list consisting of:
power, intensity, frequency, waveform, pulse envelope shape, pulse duration, pulse repetition rate, and position.

17. An apparatus to deliver energy to excitable tissue within the body of a mammal, said apparatus comprising:
a physician-operable programming device;
a patient-worn pulse generator;
an electrical driver; and
first and second energy emitters arranged in a configuration that creates a region of high energy intensity within the body remote from the emitter.

18. The apparatus of claim 17 further comprising a positioning device to adjust the location of the high intensity region.

19. A method of stimulation of excitable tissue within a body comprising:
providing a source of energy emission;
creating a region of supra-threshold energy intensity remote from the source;
maintaining a sub-threshold region intermediate the source and the supra-threshold region;
said supra-threshold region located in a region chosen from the list consisting of: a spinal cord, a spinal nerve root, a peripheral nerve, a splanchic nerve, a pudendal nerve, a sacral nerve, a vagus nerve, and an occipital nerve.

20. The method of claim 19 further comprising influencing or relieving at least partially a condition chosen from the list consisting of: a perception of pain, urinary incontinence, headache, migraine headache, sympathetic tone, psychological condition, angina symptoms, cardiac arrhythmia, satiety, endocrine production, and insulin production

21. The method of claim 19 further comprising exciting tissue chosen from the list consisting of: myocardial tissue, motor function nerves, and tissue within an organ.
